(19) United States
(12) Patent Application Publication
(10) Pub. No.: US 2007/0043071 A1
(43) Pub. Date: Feb. 22, 2007

(54) Further therapeutic use of zolpidem

(75) Inventors: Ralf P. Clauss, Guidford (GB); Wally H. Nel, Springs (ZA)

Correspondence Address: RANKIN, HILL, PORTER & CLARK, LLP, 925 EUCLID AVENUE, SUITE 700, CLEVELAND, OH, 44115-1405, US

(73) Assignee: Sciencom Ltd., Thames Ditton (GB)

(21) Appl. No.: 10/557,506

(22) PCT Filed: May 19, 2004
(86) PCT No.: PCT/GB04/02172

§ 371 Date: Mar. 6, 2006

(30) Foreign Application Priority Data
May 19, 2003 (GB) 0311457.6
May 27, 2003 (US) 60/473,821

(51) Int. Cl. A61K 31/4745

(52) U.S. Cl. 514/303

(57) ABSTRACT

An imidazo[1,2-a]pyridine-3-acetamide such as zolpidem is useful in the treatment of a condition of the brain which has a lesion and exhibits diaschisis/dormant cells at the contralateral and other sites, more particularly trauma-induced injury, spinocerebellar ataxia, cerebellar or cerebral infarct and Ramsey-Hunt syndrome.

### FIELD OF THE INVENTION

This invention relates to a new therapeutic use of zolpidem.

### BACKGROUND OF THE INVENTION

WO96/31210 discloses the use of imidazo[1,2-a]pyridine-3-acetamide derivatives, and in particular the anti-insomnia drug zolpidem, for the treatment of neuropsychiatric syndromes associated with dysfunction of the neural circuits of the basal ganglia. This use is based on the observation of the efficacy of zolpidem in the treatment of Parkinson's disease. It is reported that both the symptoms of PD (akinesia and rigidity) and obsessive-compulsive symptoms (cessation of verbal iterations) were improved.

More recently, Clauss et al, S. Afr. Med. J. (2000 January), 90(1):68-72, describes a semi-comatose patient who exits his permanent vegetative state after application of zolpidem, and reverts to it when drug action subsides. This phenomenon was further investigated in animals and ascribed to GABA(A) omega 1 receptor-specific effects in the primate brain; see Clauss et al., Arznein.-Forsch./Drug Res. (2001) 51(11):619-622.

Mayr et al, Eur. J. Neurol. (2002) 9(2)3:184-185, reports the use of zolpidem in progressive supranuclear palsy.

A further study by Clauss et al, reported in Arzneimittal Drug Research, December 2002, the contents of which are incorporated herein by reference, describes cerebral blood perfusion after treatment with zolpidem and flumazenil, in the baboon. The results of this study show that flumazenil attenuates the influence of zolpidem on the abnormal baboon, i.e. the asymmetric perfusion pattern due to the abnormality.

The concept of diaschisis was first described by Von Monakow in the early 20th century. It offers an explanation for the phenomenon of acute phase central nervous system disorder symptoms that are more extensive and of a different nature to those of the chronic phase. In a particular case, commonly seen in brain perfusion studies of cerebral stroke patients, there is a decreased blood flow in parts of the brain such as the normal cerebellum contralateral to the cerebral hemisphere injured by the stroke; this example is called crossed cerebellar diaschisis (CCD).

The onset of diaschisis can be instantaneous or it can occur within hours and can reverse spontaneously within days or years. The underlying pathogenesis of diaschisis is not clear. Implicated is a trigger resulting in a neurophysiological shutdown and decreased cerebral blood flow of uninjured brain distant from the actual site of brain damage.

Diaschisis has been reported in brain injury and in various central nervous system diseases. The incidence of diaschisis in stroke has been reported to be around 45%. Diaschisis may play a role in coma. It has been shown that traumatic brain injury is followed by a metabolic diaschisis which is related to the degree and extent of behavioural deficits. It appears that CCD is seen more often with focal cortical injuries and is more pronounced with severe brain lesions. Diaschisis can give rise to impaired consciousness and its reversal is associated with recovery of impaired function. Some authors have suggested that spontaneous reversal of diaschisis may play a role in recovery from stroke.

### SUMMARY OF THE INVENTION

The present invention is based on the surprising discovery that zolpidem and related compounds, such as those described in WO96/31210, have utility in treating conditions of the brain which exhibit diaschisis.

Based on the discovery reported herein, it is postulated that brain injury triggers a set of events that can result in a state of dormancy of normal neuronal tissue at a site, close to or (as in classical diaschisis) removed from the brain injury site. The symptomatology that is then observed in brain-injured patients is a combined symptomatology of dormant viable brain tissue and dead, non-viable brain tissue. The reversal of dormancy or diaschisis, or of non-functionality induced by ischaemia or post-ischaemia, in viable neuronal tissue after administration of zolpidem can result in reversal of brain injury effects. This effect can occur in areas of classical diaschisis and in others that may not previously have been recognised, as in what may now be termed ipsilateral diaschisis etc. As reported in a particular study (below), the diaschisis after stroke could be reversed by zolpidem, and there was improved coordination that enabled the patient to use scissors.

Without wishing to be bound by theory, it appears that the majority of brain injuries or brain pathologies have associated with them a neural dormancy or diaschisis that probably has its roots in a neuroprotective reaction of the brain during brain damage. Dormancy results in a clinical presentation that is actually worse than would be expected from the lesion alone (i.e. the brain lesion without the associated dormancy).

Dormancy or hibernation of myocardium after an ischaemic insult is a well-known phenomenon in the heart. Hibernating myocardium is non-functional but fully viable. When blood supply is re-instated after bypass surgery, hibernating myocardium becomes functional again. Similar to myocardial tissue, brain dormancy appears to occur with most forms of ischaemic brain injury or other forms of brain damage. Its reversal explains the wide efficacy of zolpidem in unrelated brain injuries, from genetic disorders such as spinocerebellar ataxia type II, to stroke and traumatic brain injury.

Brain dormancy is most likely concurrent with a structural change or folding of the complex GABA receptor molecule. This state can be at least partially reversed by zolpidem's selective GABAergic stimulation of, in particular, the omega 1 receptors.

The benefit of zolpidem in brain-injured patients is transient and it occurs for the duration of drug action only. However, after first application and proof of efficacy in an controlled environment, it could be used daily for many years in brain-injured patients, without adverse effects. Effects of the drug may remain potent even after many years of constant treatment.

It appears that zolpidem reverses symptoms due to brain dormancy but does not change those due to necrotic or scarred brain tissue. Hence the clinical effect that can be expected from the drug depends on the size and location of the brain dormancy area that can be reversed.

There is increasing evidence for an important role of zolpidem in the treatment of the sequelae of a wide range of brain pathology, based on its reversal of dormant neural tissue after brain damage. A large number of brain-injured patients may benefit from this treatment.

### DESCRIPTION OF PREFERRED EMBODIMENTS

Patients who may benefit from treatment according to the invention include those having a trauma-induced injury, but who do not necessarily exhibit akinesia or tremor, as in Parkinsonism. In particular, the patient may have lost cognition, e.g. have had a cerebellar or cerebral infarct such as in stroke. The patient may exhibit ataxia, e.g. spinocerebellar ataxia, or other symptoms related to cerebral ischemic injury. Other conditions are ruptured brain aneurism and intracerebral bleed. Alternatively or in addition, the patient may exhibit one or more of strabismus, salivation and muscle spasm, or impaired swallowing, smell or taste, or require long-term rehabilitation, e.g. over a period of one month, one year or more.

Also exemplified (below), the present invention allows the treatment of Ramsay-Hunt syndrome. Ramsay-Hunt syndrome is a complication of Herpes Zoster infection of the geniculate (facial) ganglion with a typical vesicular zoster eruption in the external auditory meatus. According to some authors, many cases previously described as the Ramsay-Hunt syndrome, as well as other hitherto unclassified system degenerations associated with myoclonus epilepsy, are examples of myoclonus, epilepsy and ragged red fibres (MERRF).

Vascular and multi-infarct dementia, and Bell's palsy e.g. of cerebral origin, may also be treated by this invention. Such conditions are characterised by areas of diaschisis/dormancy in the brain.

Such conditions can be treated according to the invention, so that the patient has increased mobility and functionality. The patient may exhibit some evidence of regeneration. As in the case of spinocerebellar ataxia, the present invention may provide the first effective treatment.

Areas of diaschisis or dormancy may be identified, e.g. by brain SPECT (single photon emission computed tomography).

For use in the invention, zolpidem (which is used herein for the purposes of illustration) will usually be given by a method that avoids undue sedation. Two examples are a small but relatively more frequent dose by the sublingual route for rapid absorption or a depot providing sustained release that avoids a peak of absorption that usually follows the use of tablets or capsules.

The dosage of zolpidem may be, for example, 1 to 100 mg of the drug per day. Suitable formulations, routes of administration and dosages will be evident to one of ordinary skill in the art, and will be chosen according to the usual factors, such as the potency of the drug, the route of administration, the severity of the condition, the state of the patient etc.

The present invention is based on the following illustrative Examples.

### EXAMPLE 1

A male patient was prescribed 10 mg zolpidem for treatment of his insomnia. He had suffered a stroke several years before and presented with left-sided paraplegia since the onset of his stroke. His cognition was still normal but he had some aphasia and decreased proprioseption that was evident from his inability to use scissors with his right hand. The clinical and neurological features of the patient were unremarkable apart from the above. The patient was investigated by ^{99m}Tc HMPAO Brain Spect before and after zolpidem application.

Two brain SPECT studies were completed on different days. The first study was completed in the normal baseline state and the second study was performed 1 hour after application of 10 mg zolpidem on the following day. The imaging was started 30 minutes after intravenous injection of 900 MBq ^{99m}Tc HMPAO, using a dual head SOPHY DST XLi gamma camera. Acquisition parameters were 64 angular views over 360° at 45 seconds per view. Ultra high resolution fanbeam collimation without zoom was used and a 20% symmetrical window over 140 KeV. The images were reconstructed using a Metz prefilter. Transaxial, sagital and coronal slices were constructed without attenuation or scatter correction. The images before and after application of zolpidem were assessed in comparative transaxial slices and in different segments of the brain.

Results of the two studies appeared similar in most respects. There was no tracer uptake in a large part of the right cerebral hemisphere. As may be expected in stroke, the non-zolpidem baseline study showed a crossed cerebellar diaschisis with decreased tracer uptake in the left cerebellum. However, although the area affected by the stroke did not change after zolpidem application, the crossed cerebellar diaschisis was reversed and cerebellar ^{99m}Tc HMPAO uptake normalised. There were no marked clinical changes after zolpidem, except that the patient was able to use scissors for the next few hours until drug action subsided.

### EXAMPLE 2

There is currently no effective pharmacologic treatment for spinocerebellar ataxia. This Example reports a family of five patients, four of which improved clinically within 1 hour of 10 mg zolpidem application. Spinocerebellar Ataxia Type 2 was confirmed by molecular analysis. DNA, analyzed for CAG repeat expansions in the SCA1, −2, −3, −6 and −7 genes, revealed expansion of CAG repeats at the SCA2 locus.

P1: 49 year male with titubation, dizziness and loss of balance from age 34. Deteriorating speech and handwriting. Cerebellar signs included moderate gait ataxia, intention tremor, dysdiadochokinesis and titubation. Deep tendon reflexes were all brisk. After zolpidem, ataxia, intention tremor and titubation improved moderately.

P2: 37 year male with loss of balance and deterioration of handwriting since age 25. He had bilaterally brisk tendon reflexes, ataxia, intention tremor, dysdiadochokinesis and titubation. After zolpidem ataxia, intention tremor and titubation improved. P3: 45 year male with speech incoordination since age 30. Current explosive speech and severe dysarthria. His handwriting and speech continue to deteriorate. Tendon reflexes were bilaterally brisk. He had titubation, intention tremor, disdiachokinesis and gait ataxia. After zolpidem, ataxia, intention tremor and titubation improved moderately.

P4: 22 year female who developed loss of balance at age 18 with subsequent speech deterioration. Occasional titubation. She was clinically depressed and dull in emotion, only responding to instructions. She had gait ataxia and intention tremor. There was no improvement after zolpidem.

P5: 24 year female with leg weakness and loss of balance. Speech deteriorated from age 22. Cerebellar signs included ataxia, intention tremor and dysdiachokinesis with intermittent titubation. Zolpidem slightly improved ataxia, intention tremor and titubation.

99 mTc HMPAO Brain SPECT showed subnormal tracer concentration in the vermis or a cerebellar hemisphere in all patients. One patient (P2) showed markedly decreased uptake in the left thalamus and cerebellum that normalized after zolpidem.

### EXAMPLE 3

Zolpidem was used for the therapy of Ramsay-Hunt syndrome in a 60-year old patient who had suffered from the condition for two months before zolpidem treatment. On treatment, the following features improved. He was able to drink fluids directly from a cup, rather than through a straw and the tonus of his facial nerves improved. Also, as part of the syndrome he could not close his left eye. After zolpidem he could.

Cortical dysfunction is a prominent clinical feature in MERRF. Taking into consideration that Ramsay-Hunt syndrome is possibly a variant of MERRF, the efficacy of zolpidem that was observed is most likely due to the reversal of dormant neural tissue or diaschisis components that may be associated with brain damage in MERFF.

### EXAMPLE 4

A 54 year old male patient presented with impaired hearing after a subachnoid haemorrhage some two years prior to his appointment. He had had a previous stroke more than a decade before the event, when aged 42, with partial loss of function in his left arm and leg. He was coping well in his environment and was leading a near normal life.

After his subarachnoid haemorrhage, he could not swallow, hear or speak for several weeks. He had poor balance and some cognitive and memory impairment with some personality changes. There was visual field loss bilaterally. The eyes remained normal but the patient could not read and object recognition decreased. There was no diplopia, hallucination, sphincter loss, tinnitus, vertigo or voice change.

Swallowing improved 8 weeks after the haemorrhage. Although the patient started to speak again some time later, there was a difficulty with articulation, expression and understanding. Several months after the haemorrhage, he moved back into his own home and lived there with his carer. He did many things there himself, including cooking, tooth brushing, washing and ironing.

Communication and hearing remained a problem to the patient and he also complained of insomnia. Hearing was slightly impaired and he could not understand the meaning of words. His speech was also impaired and he was attending regular speech therapy sessions to improve his speaking.

After extensive counselling and advice to the patient and his family, a 10 mg test dose of zolpidem was given. The patient was then examined for any changes in hearing. Results were compared to the baseline state with no zolpidem. The patient was also evaluated by the speech therapist before and after zolpidem.

He was then booked for Brain SPECT investigation before and after application of 10 mg Zolpidem. Two brain SPECT studies were completed on different days. The first study was completed during the baseline state and the second study was performed 1 hour after application of 10 mg zolpidem. (It was noted previously that the patient's maximum response to zolpidem was one hour after drug application).

Although the patient was slightly sleepy after 10 mg zolpidem, his hearing improved as shown pre and post-zolpidem audiograms. Furthermore his speech ability improved markedly. These improvements remained for the duration of drug action, even after halving the zolpidem dose to reduce sleepiness. On Brain SPECT there was a defect noted in the right parietal aspect of his brain that remained unchanged after zolpidem but there was also decreased tracer uptake in the left thalamus and medial frontal regions bilaterally that improved after zolpidem.

### FURTHER EVIDENCE

A previous study has showed that CCD is associated with alterations in the GABA(A)/BZR complex and that reorganisation of GABA-mediation and glucose metabolism occurs in the cerebellum following cortical injury (Niimura et al, 1999). In another study, diaschisis was associated with a down-regulation of GABA receptor binding and an altered composition of GABA receptor subunits (Witte et al., 1997). These findings support the present invention. They support the short and long-term modulating role of GABA receptors after brain injury and the theory that symptomatology after brain injury could be influenced by transient or permanent up-or-down regulation of GABA receptors and their subunits. They also support the view that GABA mechanisms are involved in diaschisis. The observed effects of the omega 1-specific drug zolpidem in brain injury and in diaschisis support the theory that omega 1 receptors in particular play an important role in brain injury symptoms.

Further, the study by Clauss et al (2002), briefly discussed above, supports the present invention when it is realised that the subject exhibited diaschisis. The effect of zolpidem in the brain-abnormal baboon (asymmetric increased perfusion) and in the brain-damaged human (semi-comatose to conscious) clearly demonstrates that zolpidem exerts its influence most profoundly in the abnormal damaged brain and that zolpidem shows beneficial effects in particular in the presence of a pathological brain state.

The above evidence indicates a role for GABA and GABA-dependent systems in brain injury and ultimately coma. When zolpidem is applied some time after brain injury, there is an improvement in the clinical features caused by the brain injury. Concurrent changes in brain perfusion and metabolism are usually detected on 99 mTc HMPAO Brain SPECT. The action is highly specific and it involves in particular omega 1 GABA systems. For instance, when the semi-comatose patient received the non-selective benzodiazepine diazepam instead of zolpidem for imaging studies, he was not awakened.

1. Use of an imidazo[1,2-a]pyridine-3-acetamide for the manufacture of a medicament for use in the treatment of a condition of the brain which has a lesion and exhibits diaschisis/dormant cells at the contralateral and other sites.

2. Use according to claim 1, wherein the imidazo[1,2-a]pyridine-3-acetamide is zolpidem.

3. Use according to claim 1, wherein the subject of treatment has a trauma-induced injury.

4. Use according to claim 1, wherein the subject of treatment exhibits ataxia.

5. Use according to claim 4, wherein the subject of treatment exhibits spinocerebellar ataxia.

6. Use according to claim 1, wherein the subject of treatment exhibits a condition selected from strabismus, salivation, muscle spasm and impaired swallowing, smell or taste, hearing or speaking, or requires long-term rehabilitation.

7. Use according to claim 1, wherein the subject of treatment has a ruptured brain aneurism or intracerebral bleed.

8. Use according to claim 1, wherein the subject of treatment has a cerebellar or cerebral infarct.

9. Use according to claim 1, wherein the subject of treatment has lost cognition.

10. Use according to claim 1, wherein the condition is ischaemic or post-ischaemic.

11. Use according to claim 1, wherein the condition is Ramsay Hunt syndrome.

12. A method of treating a subject having a brain lesion and diaschisis, dormancy and/or non-functionality in viable neuronal tissue, the method comprising administrating a medicament to the subject comprising an imidazo[1,2-a]pyridine-3-acetamide.

13. The method according to claim 1 wherein the imidazo[1,2-a]pyridine-3-acetamide is zolpidem.

14. The method according to claim 1 wherein the brain lesion is trauma-induced.

15. The method according to claim 1 wherein the subject exhibits ataxia.

16. The method according to claim 15 wherein the subject exhibits spinocerebellar ataxia.

17. The method according to claim 1 wherein the subject exhibits a condition selected from strabismus, salivation, muscle spasm and impaired swallowing, smell or taste, hearing or speaking, or requires long-term rehabilitation.

18. The method according to claim 1 wherein the subject has a ruptured brain aneurism or intracerebral bleeding.

19. The method according to claim 1 wherein the subject has a cerebellar or cerebral infarct.

20. The method according to claim 1 wherein the subject of treatment has lost cognition.

21. The method according to claim 1 wherein the non-functionality is induced by ischaemia or post-ischaemia.

22. The method according to claim 1 wherein the subject has Ramsay Hunt syndrome.
